(19) United States
(12) Patent Application Publication
(10) Pub. No.: US 2014/0343204 A1
(43) Pub. Date: Nov. 20, 2014

(54) Esters, Hydrogenated Derivatives Thereof, and Processes for Producing the Same

(71) Applicants: Didier Naert, Bruxelles (BE); Allen D. Godwin, Seabrook, TX (US)

(72) Inventors: Didier Naert, Bruxelles (BE); Allen D. Godwin, Seabrook, TX (US)

(21) Appl. No.: 14/233,207

(22) PCT Filed: Jun. 7, 2012
(86) PCT No.: PCT/US12/41215

§ 371 Date: Apr. 17, 2014

Related U.S. Application Data
(60) Provisional application No. 61/523,706, filed on Aug. 15, 2011.

(51) Int. Cl. C07C 67/08; C08K 5/12; C07C 67/303

(52) U.S. Cl. CPC C07C 67/08; C07C 67/303; C08K 5/12
USPC 524/285; 560/127; 106/316

(57) ABSTRACT

Processes for the production of compositions including esters and their hydrogenated derivatives are provided. The esters and the hydrogenated derivatives may be produced with among other things alcohols that are derived from biological sources such as photosynthetic microorganisms. The esters and/or the hydrogenated derivatives may be employed as plasticizers for polymeric compositions.

### CROSS REFERENCE TO RELATED APPLICATIONS

This application claims the benefit of Ser. No. 61/523,706, filed Aug. 15, 2011, the disclosure of which is incorporated by reference in entirety.

### FIELD OF THE INVENTION

The present invention relates to processes for the production of compositions comprising esters. The esters may be produced with among other things alcohols that are derived from biological sources. The esters may optionally be hydrogenated to produce hydrogenated derivatives. Either the ester and/or the hydrogenated derivative may be employed as a plasticizer for polymeric compositions.

### BACKGROUND

Esters are typically produced by the reaction of an alcohol with a carboxylic acid or a carboxylic acid anhydride. In many instances, one or more of the starting materials may be a mixture. The carboxylic acid may be a mono or a polycarboxylic acid or the anhydride thereof. Plasticizer esters are generally produced from polycarboxylic acids or the anhydrides thereof and in particular from phthalic anhydride, cyclohexanoic dicarboxylic acid or its anhydride, adipic acid or its anhydride, or trimellitic acid or its anhydride. Esters of benzoic acid, such as isononyl benzoate or isodecyl benzoate, are examples of plasticizer mono-esters. Esters may be produced from any alcohol. However, plasticizer esters are generally produced from C₄ to C₁₃ alcohols, in particular, C₆ to C₁₃ alcohols, and more typically, C₈ to C₁₀ alcohols, including mixtures of any of the aforementioned alcohols.

The production of plasticizer esters is described in, for example, U.S. Pat. Nos. 5,324,853; 5,880,310; 6,310,235; and 6,355,817. Typically, the alcohols referenced above are supplied from and the product of a multi-step process that begins with lower linear alpha olefins derived from a petroleum source oligomerized to higher olefins, followed by at least one hydroformylation step, and subsequently hydrogenation to produce a mixture of alcohols and other products.

Alcohols may also be derived from other starting materials including biological sources. For example, U.S. Pat. No. 1,993,736 discloses, among other things, processes for the production of organic acid esters such as lauryl phthalates made from aliphatic alcohols derived from the catalytic hydrogenation of coconut oil acids. The organic acid esters may be used as softeners and plasticizers for cellulosic plastic compositions. Similarly, U.S. Pat. No. 1,993,737 discloses among other things the production of polycarboxylic acid esters (e.g., didecyl phthalate) of n-decyl alcohol. The alcohols may also be obtained from the hydrogenation of coconut oil. The esters may be used as plasticizers for cellulose derivatives to produce coatings and plastic compositions. In related areas, U.S. Pat. No. 1,972,091 discloses, among other things, the production of a cellulose derivative compositions using phthalates (e.g., dilauryl phthalate, lauryl butyl phthalate) made from alcohols having 6-15 carbon atoms. The cellulose derivative materials may be used as lacquers, molding compositions, or materials used in the preparation of coated fabrics.

Plasticizer esters may also be hydrogenated. For example, U.S. Pat. No. 2,070,770 discloses among other things dialkyl phthalates having alkyl groups obtainable from coconut oil that have been hydrogenated over a nickel metal catalyst to produce “hexahydrophthalates” also known as cyclohexanedicarboxylic acid esters or cyclohexanoates. In some parts of countries and in some countries, cyclohexanoates have wider industry acceptance because of regulatory schemes.

In recent years, chemical products derived from biological sources have become more interesting because the increased availability of programs that seek to find alternatives to petroleum products and the desire to produce products that further advance the effort to produce chemical products with greater sustainability. Sustainability encompasses producing products in ways that augment and consider economic growth, social development and environmental concerns. For example, there has been much excitement and interest regarding biodiesel. Biodiesel generally refers to mono-alkyl esters of long chain fatty acids that may be derived from rapeseed, soybean, mustard, flax, sunflower, canola, palm oil, hemp, jatropha, waste vegetable oils, other waste products produced by meat processing, sugar cane, and certain cellulosic materials. There have also been studies to support the proposition that biodiesel fuel could commercially be produced from algae oil.

Therefore, embodiments of the invention described and claimed herein address this need by providing esters and hydrogenated derivatives of those esters made from alcohols that are derived from biological sources. The esters and hydrogenated derivatives of those esters may be used as plasticizers for polymeric compositions. The polymeric compositions may offer greater efficiencies in formulating as compared to current commercial offerings, be labelled as “green” products offering greater acceptance in more industries and countries, and/or offer environmental credits from programs that seek to provide incentives to brings these products to market.

### SUMMARY

In a class of embodiments, the invention provides for a process for the production of an ester, comprising:

(a) obtaining at least one C₄ to C₁₃ alcohol derived from one or more photosynthetic microorganisms; and
(b) contacting and reacting at least one carboxylic acid or at least one carboxylic acid anhydride with the at least one C₄ to C₁₃ alcohol to produce a product comprising the ester resulting from the reaction.

In another class of embodiments, the invention provides for a process for the production of a cyclohexanepolycarboxylic acid ester, comprising:

(a) obtaining at least one C₄ to C₁₃ alcohol derived from one or more photosynthetic microorganisms;
(b) contacting and reacting at least one carboxylic acid or at least one carboxylic acid anhydride with the at least one C₄ to C₁₃ alcohol to produce a product comprising an ester; and
(c) hydrogenating the ester to produce a second product comprising the cyclohexanepolycarboxylic acid ester.

In yet another class of embodiments, the invention provides for a plasticizer composition comprising an ester of at least one C₄ to C₁₃ alcohol derived from one or more photosynthetic microorganisms and at least one carboxylic acid or at least one anhydride; wherein the plasticizer composition has a biobased content of 60.0 wt % or more (ASTM D6866-10) based upon the total weight of the plasticizer composition.

Additional embodiments are disclosed and claimed herein.

### DETAILED DESCRIPTION

Before the present compounds, components, compositions, and/or methods are disclosed and described, it is to be understood that unless otherwise indicated this invention is not limited to specific compounds, components, compositions, reactants, reaction conditions, or the like, as such may vary, unless otherwise specified. It is also to be understood that the terminology used herein is for the purpose of describing particular embodiments only and is not intended to be limiting.

It must also be noted that, as used in the specification and the appended claims, the singular forms “a,” “an” and “the” include plural referents unless otherwise specified.

Esters are generally produced by the reaction of an alcohol with a carboxylic acid or a carboxylic acid anhydride. In many instances, one or more of the starting materials may be a mixture. The carboxylic acid may be a mono or a polycarboxylic acid or the anhydride thereof. Plasticizer esters are generally produced from at least one alcohol and polycarboxylic acids or the anhydrides thereof and in particular from phthalic anhydride, maleic anhydride, cyclohexanoic dicarboxylic acid or its anhydride, adipic acid or its anhydride or trimellitic acid or its anhydride. Esters of benzoic acid, such as isononyl benzoate or isodecyl benzoate, are examples of plasticizer mono-esters.

Examples of esters include phthalate esters such as dioctyl phthalate, di-isononyl phthalate, di-isodecyl phthalate, di-isooctyl phthalate, di-isoheptyl phthalate, di-2ethyl hexyl phthalate, and mixtures thereof.

Esters may be produced from any alcohol, but plasticizer esters are generally produced from C₄ to C₁₃ alcohols, in particular C₆ to C₁₃ alcohols, typically, C₆ to C₁₁ alcohols, more typically, C₈ to C₁₀ alcohols, including mixtures of any of the aforementioned alcohols. The production of esters and in particular plasticizer esters is described in, for example, U.S. Pat. Nos. 5,324,853; 5,880,310; 6,310,235; 6,355,817; U.S. Patent Application Publication No. 2010/0130767, and WO 2008/110305.

Several classes of embodiments of the invention relate to improvements in or relating to esterification processes and in particular to esterification processes of acids and anhydrides with alcohols derived by in whole or in-part from biological sources, for example, photosynthetic microorganisms, particularly for the production of esters of phthalic acid and anhydrides, adipic acid, trimellitic, cyclohexane carboxylic acids and benzoic acid and C₄ to C₁₅. The esters may optionally be hydrogenated to produce hydrogenated derivatives, for example, cyclohexanedicarboxylic acid esters or also known as cyclohexanoates. Such esters or hydrogenated derivatives are particularly useful as plasticizers for polyvinyl chloride (PVC), polyurethane, and acrylic compositions.

### Esterification

Several embodiments of the invention provide for a process for the production of C₄ to C₁₅ esters, alternatively, C₆ to C₁₂ esters, and alternatively, C₈ to C₁₀ esters, by the esterification of a carboxylic acid or its anhydride with at least one C₄ to C₁₅ alcohol derived from biological sources.

The at least one alcohol is preferably at least one C₄ to C₁₅ alcohol, alternatively, at least one C₆ to C₁₂ alcohol, and alternatively, at least one C₈ to C₁₀ alcohol, and mixtures thereof, derived from biological sources, for example, photosynthetic microorganisms. Examples include hexanol, heptanol, isoheptanol, 2-ethyl-hexanol, octanol, nonanol, isononanol, decanol, and mixtures thereof, optionally branched or linear. Examples also include mixtures of branched chain nonanols, linear and branched chain decanols including 2-propyl-heptanol, normal and/or branched undecanol and tridecanol. In an embodiment, the at least one alcohol comprises n-hexanol, n-octanol, n-decanol, and mixtures thereof. In another embodiment, the at least one alcohol comprises a mixture of C₆, C₈, and C₁₀ alcohols, C₈ and C₁₀ alcohols, or C₁₀ and C₁₂ alcohols.

As used herein, “photosynthetic microorganism” may refer to any prokaryotic (having properties similar to bacteria) or eukaryotic (having properties similar to plants and animals) single-celled or multi-cellular organisms that can perform photosynthesis. Organization of cell types include colonial, capsoid, coccoid, palmelloid, filamentous, and parenchymatous. Photosynthetic microorganisms include but are not limited to eukaryotic microalgae and prokaryotic organisms generally known as photosynthetic bacteria.

Eukaryotic microalgae or microphytes include but are not limited to species of green algae (Chlorophyceae), yellow-green algae (Xanthophyceae), golden algae (Chrysophyceae), brown algae (Phaeophyceae), red algae (Rhodophyceae), diatoms (Bacillariophyceae), and “pico-plankton” (Prasinophyceae and Eustigmatophyceae).

Prokaryotic organisms include but are not limited to cyanobacteria, green sulfur bacteria, purple sulfur bacteria, purple nonsulfur bacteria, and green nonsulfur bacteria.

Photosynthetic microorganisms include those organisms occurring and found in nature as well as those organisms that have been modified directly or have at least one modification within the organisms' genetic lineage.

As used herein, “derived from one or more photosynthetic microorganism(s)” is intended to exclude derivations from fossilized organic materials such as, for example, natural gas and petroleum or crude oil.

The production of photosynthetic microorganisms and the use of these microorganisms to produce alcohols are known and an example may be found in WO 2010/068821. General methods for harvesting, producing, and processing photosynthetic microorganisms may be found in, for example, WO 2010/059801, WO 2009/039333, WO 2004/020810, U.S. Patent Application Publication Nos. 2010/0055765, 2009/0321349, 2009/0203116, 2009/0181440, 2009/0077864, and 2009/0056201.

A C¹⁴ dating process described in ASTM D6866-10 may be used to distinguish the alcohols as described above from those alcohols derived from petroleum or other sources. In particular, in a class of embodiments, the at least one alcohol derived from photosynthetic microorganisms will have a C¹⁴/C¹² ratio of 1:10¹² in accordance with ASTM D6668-10. In contrast, for reference, alcohols derived from petroleum or crude oil will generally have C¹⁴ content much lower approaching zero or essentially zero. For C¹⁴ references, ASTM D6668-10 is applied unless otherwise specified.

The at least one alcohol described above may be used with commercially available alcohols. Examples include hexanol, heptanol, isoheptanol, 2-ethyl-hexanol, nonanol, isononanol and mixtures of branched chain nonanols, linear and branched chain decanols, including 2-propyl-heptanol such as those available from Evonik and BASF, normal and/or branched undecanol and tridecanol.

In a class of embodiments, the process is particularly useful in esterifications catalyzed by titanium, zirconium, or tin organometallic catalysts.

In a class of embodiments, the mixture of reactants is caused to react by an esterification catalyst by heating the mixture containing the catalyst at a temperature in the range of from 200° C. to 250° C., more preferably the reaction is performed at a temperature in the range from 205° C. to 225° C. The reaction mixture is also stirred at this temperature. Titanates are preferred catalysts and they typically become active at around 210° C. and reaction water is generated rapidly at this temperature.

In other embodiments, acid catalysts, such as, for example, sulphuric acid, p-toluene sulfonic acid, etc. may be used.

In another embodiment, a greater stoichiometric excess of the alcohol is employed as this decreases the boiling point of the mixture which enables the reaction to be performed at higher pressures than the conventional pressures and at lower temperatures. In several embodiments, “excess” refers to any value more than the stoichiometric amount to run a chemical reaction, for example, an esterification process. In several embodiments, “greater stoichiometric excess” refers to 1% or more, alternatively, 2% or more, alternatively, 5% or more, alternatively, 10% or more, alternatively, 15% or more, alternatively, 20% or more, and alternatively, 25% or more, of the “excess” as defined above in mol %. These conditions enable effective removal of the water formed in reaction by boil up of the alcohol water mixture and it further reduces the carry over in the reflux of the acid and/or the ester formed in the reaction. The excess alcohol also pushes the reaction towards completion. In an embodiment, 20 to 35 mol % excess alcohol is used in the process as defined above. In a further embodiment, the invention is applied to esterification reactions in which the alcohol has been purified by treatment with sodium borohydride. It is however also applicable to reactions in which the alcohol is purified by hydrofining or to processes in which mixtures of such alcohols are used.

The temperature of the mixture (“mixture temperature”) when the catalyst is introduced is determined by the operator to suit the system employed. For convenience, this temperature is designated the “predetermined mixture temperature”. The terms “predetermined mixture temperature”, “desired esterification reaction temperature”, and “initial temperature of the mixture” are used herein to indicate temperatures within temperature ranges that are known to be appropriate for a particular catalysed esterification reaction. For example, the desired esterification reaction temperature and predetermined mixture temperature will vary according to the nature of the reactants and the nature of the catalyst, and the invention does not concern these particular conditions but is concerned with the conditions that should be used depending on these particular temperature conditions.

Increasing the pressure in the reactor increases the difference in the boiling points of the reactants and the reaction product thus enhancing the ability to separate them under reflux. Furthermore, the increase in pressure ensures that the heat supplied to the reaction mixture is used for heating the reactants, rather than for vaporisation of the reactants. Within heat input limited equipment, this minimizes the time to reach the minimum desired temperature for the esterification reaction, which assures maximum achievable reaction rate. This in turn enables a reduction in reaction cycle time. In addition, the increased pressure and reduced vaporisation rate allow the predetermined mixture temperature, at which the catalyst is introduced, to be reached sooner, thereby further reducing the reaction time. Furthermore, since, during this initial stage of the reaction vaporisation of the reactants, particularly of alcohol, is substantially reduced, there is no requirement for reflux, thus avoiding the extra cooling effect of the refluxed material. Less alcohol in the vapour phase and in the overhead reflux system also means more alcohol in the reactant liquid mixture, which by its higher alcohol concentration assures a higher reaction rate again.

In some embodiments, the catalyst should be added after the mixing of the carboxylic acid or its anhydride and the alcohol and at a predetermined mixture temperature above the initial temperature of the mixture. In an embodiment, the catalyst is added to the mixture when it is at a temperature in the range from 160° C. to 220° C., more preferably from 180° C. to 200° C.

In a class of embodiments, the catalyst is generally added at a temperature below the desired esterification reaction temperature and, accordingly, after catalyst addition the temperature of the mixture is increased further to the desired esterification reaction temperature. Although the catalyst activity increases with increasing temperature, its stability decreases with increasing temperature and accordingly, depending on the nature of the catalyst, there is an optimum reaction temperature or a fairly narrow optimal temperature range. Irrespective of this temperature or range, addition of the catalyst causes the formation of more water at a faster rate which must be removed rapidly so that, with titanate catalyst, it has less chance of hydrolysing the water-sensitive catalyst. Traditionally, heat is continued to be supplied once the reaction temperature is reached, to promote the reaction, however, we have found that if the energy supplied to the reactor which maybe heat and/or the intensity of the stirring is reduced once the reaction temperature was reached the level of foaming was reduced at least to the extent that any foam produced was confined to the reactor. The degree of reduction of energy input that is required depends upon the reactants.

One or more of the starting materials, comprising the carboxylic acid or its anhydride, or the alcohol and any recycle of the excess reactant, may be preheated before being mixed with the other reactants or before being introduced into the reaction vessel, such as up to a temperature of from 100° C. to 160° C. Oxygen may be removed from one or more of them, preferably from at least one of the fresh starting materials, to improve ester product colour. This oxygen removal occurs preferably after preheating and preferably by nitrogen stripping if it concerns a liquid, or by nitrogen purging of the equipment if it concerns a solid. These pre-treatments are preferably performed in a separate vessel before the starting material is introduced into the reactor vessel. The preheating reduces the reaction batch time, and performing the preheating and/or the oxygen removal in a separate vessel further reduces the time that a particular batch occupies the reactor and therefore also the overall reaction batch time. Embodiments of the invention therefore provides a process employing a particular energy input profile. The preferred profile depends upon the nature of the reactants and the relative quantities.

A variety of heating means may be applied to the esterification reactor to provide the controlled heat input. Many processes provide heat input by circulating a heating medium through one or more heating coils provided in the reactor, preferably under the liquid level, and/or through a heating mantle around the reactor wall. In some embodiments, steam heating is more effective in transferring heat than hot oil which preferably is at a pressure sufficiently high that it condenses at a temperature above the temperature of the reaction mixture. In some embodiments, a high pressure steam at a pressure of at least 40 barg (about 600 psig) is used. The heat supplied may be controlled automatically by sensors within the reaction vessel or optionally in the heating medium circulation lines.

By applying and maintaining the pressure above atmospheric during the initial phase of the esterification reaction, water can be removed without the significant alcohol boil off that occurs in known processes which operate at atmospheric or reduced pressure in this initial phase. The use of the higher pressure reduces the need for alcohol recapture and recycle and hence increases the efficiency of the reaction. The use of increased pressure also maximizes the usefulness of the heat supply into heating the reaction mixture, and results in the optimum reaction temperature being reached in a shorter time. It also keeps the reactant concentrations in the reaction mixture at the highest possible level. Both these factors result in a faster reaction rate.

In an embodiment, the esterification process is preferably performed initially under a blanket of inert gas such as nitrogen or methane. The pressure within the reactor (before any vapour vent is opened or vacuum system is commissioned) therefore, depends upon the pressure exerted by the inert gas combined with that exerted by the vapours within the reactor, which in turn depends upon the degree of reaction and the extent to which the reactants and the products of the reaction are vaporized, which in turn depends upon the temperature of the reaction. The temperature and therefore the pressure also depends upon the extent to which materials are refluxed. It is therefore preferred that the reactor system be provided with a vent valve and also a gas supply whereby gas may be introduced to increase the pressure within the reactor.

As reactor temperature and pressure increase, a vapour cloud of condensibles, i.e., primarily water but accompanied by some of the lighter boiling reactant, develops above the reactor liquid and displaces the inert gas that filled the reactor initially. The inert gas is pushed into the overhead system, and at a certain moment, the vapour cloud of condensibles reaches the reactor overhead condenser. At that time condensation typically starts, and liquids collect in the overhead separator. Depending on the initial liquid level in the overhead separator, sooner or later the liquid will overflow and the reflux of lighter boiling reactant to the reactor may be activated. We have found there is a tendency for the temperature of the reaction mixture to drop once the reflux system is activated. This in turn leads to a reduction in the pressure within the reactor. We have found that it may be particularly beneficial to introduce uncondensible gas into the reactor at this time to bring the pressure back up to at least re-establishing the desired conditions.

The vent and the gas supply may be provided at any suitable position in the reactor system which typically comprises reactant feed means, a reactor provided with heating means, a condenser, means for the separation of condensed materials, means for the recycle of reactants and means for reaction product removal. The vent and the gas supply may be provided in the reactor or elsewhere and we prefer that they are provided at or close to the condenser where it is most effective in impairing or stopping condensation which is typically still undesired at that time.

The reactor is provided with a mixer and, in the preferred reaction cycle, the fresh alcohol feed is introduced into the reactor until a minimum level is reached. At this stage, the mixer is activated and introduction of the acid, such as benzoic acid, is instigated; further alcohol consisting of fresh alcohol or recycle alcohol may also be introduced. Reactor heating may be implemented at this time, preferably as soon as the liquid level in the reactor reaches the surface of the heating equipment.

The reactor system is also provided with means for the introduction of the catalyst into the reaction mixture, preferably, introducing the catalyst below the surface of the stirred reaction mixture. The means must be such that the catalyst can be introduced into the reactor when it is under superatmospheric pressure. It is therefore preferred that the catalyst be injected into the reactor by means of pressure of the inert gas that is used as the blanket for the reaction. It is also preferred that after the catalyst is injected, the catalyst injection system be flushed with at least one of the reactants. In particular, where the esterification comprises the reaction of an acid or anhydride with an alcohol, we prefer that the catalyst injector be flushed with the alcohol. When the catalyst is water sensitive, such as with titanium catalyst, it is preferred that the reactant used for flushing has a low water content, such as at most 500 ppm by weight, preferably at most 200 ppm by weight, most preferably at most 100 ppm by weight. After introduction of the catalyst the temperature is raised to the reaction temperature, typically from 210° C. to 225° C. In this embodiment, once the reaction temperature is reached, the energy input to the reaction mixture is reduced by lowering the heat input and, optionally also reducing the degree of stirring.

The techniques of the present invention are particularly useful when used in combination with other techniques that are known for improving the efficiency of esterification reactions. In particular, the techniques may be used with other techniques that are known for minimising contact between water and the esterification catalyst. For example, the reaction system may include a reflux drier column, such as is described in U.S. Pat. No. 5,324,853. A reflux column or drier serves to heat and dry the condensed alcohol as it is being refluxed for recycle to the reaction, preferably, by using hot vapours from the reactor as heating medium. As an alternative, the cold condensed alcohol from the overhead collector may be heated and flashed to remove most of the water as vapour, and the flashed liquid may then be refluxed to the reactor, optionally, routed through a reflux drier column to achieve even lower water levels. Another useful technique is described in WO2008/110306, in which the catalyst is introduced into the reaction mixture below the surface of the liquid reaction mixture. This may be accomplished by the injection of the catalyst through a probe whose opening is below the surface of the liquid reaction mixture. In this way, contact between the catalyst and any water rich vapour in the atmosphere above the liquid reaction mixture may be minimised and the catalyst stability preserved. In some embodiments, it is also preferred that after the catalyst is injected, the catalyst injection system be flushed with at least one of the reactants.

We have found that reflux drying improves reaction batch time because of the lower water content and the higher temperature of the reflux. This reduces the amount of heat required to revaporise the water in the reflux and required to heat the colder reflux up to the reaction temperature. We have also found that a larger size reflux drier column allows a steeper pressure profile due to the lower pressure drop in the vapour flowing to the reactor overhead system. We have also found that, in case the reflux drier column cross section is causing an excessive pressure drop, a partial vapour bypass over the drier column may alleviate this problem and help reaching higher reactor productivities whilst the reflux continues to be adequately dried.

In an embodiment, we prefer that the esterification processes be performed in the manner described in WO 2008/110305, wherein the esterification recipe and the feed pre-treatment are optimised in order to optimise the reaction rate and to reduce reaction time. A particularly preferred reaction cycle for the production of esters and in particular plasticizer esters comprises this feed recipe adjustment and pre-treatment followed by the employment of the reaction process of embodiments of the present invention, followed by the neutralisation technique of WO2006/125670 and the purification techniques of WO2005/021482. The preferred cycle of the present invention depends upon the nature of the reactants and the catalyst. In the preferred cycle, alcohol is preheated to a temperature in the range from 100° C. to 160° C. This preferred preheating temperature is grade dependent, because of the change in boiling point. Excessive preheating is to be avoided in order to keep alcohol vapour losses from the preheating step within acceptable limits. For C₇ alcohol we prefer to preheat from 100° C. to 115° C., for C₉ and C₁₀ alcohol we prefer from 130° C. to 150° C., and for C₁₁ or higher, such as isotridecyl alcohol, we prefer from 130° C. to 155° C. or even 160° C. The preheated alcohol is then preferably added to a reaction vessel that is blanketed with an inert gas preferably nitrogen or methane and is heated at a temperature in the range from 120° C. to 150° C. or 160° C. and is at atmospheric pressure. Maximum heat input to the reactor is preferably applied as soon as possible. The acid or acid anhydride is then added at a temperature in the range from 135° C. to 160° C. or even up to 180° C. The content of the reaction vessel is then rapidly heated to the predetermined mixture temperature at which the catalyst is added.

Until the desired esterification reaction temperature is reached, the pressure of the reaction vessel should be maintained at a level sufficient to distil off the water whilst preventing significant alcohol boiling while forming an ester from the reactants. The pressure of the reaction vessel is generally adjusted continually to ensure continuous vaporisation and removal of water. Typically, the initial reaction pressure is close to atmospheric pressure, for example 1 to 2 bara (101.3 to 202.6 kPa), and moves through a maximum, when the desired esterification reaction temperature or the lower end of the optimal range is reached, of for example 1.5 to 2.5 bara (152.0 to 253.2 kPa), and then reduces toward an increasing vacuum as the reaction proceeds. Preferably, the final reaction pressure ranges from 2 bara (202.6 kPa) to 100 mm Hg absolute (13.3 kPa). More preferably, the final reaction pressure ranges from 1.0 bara (101.3 kPa) to 150 mm Hg absolute (20 kPa). Most preferably, the final reaction pressure ranges from 190 mm Hg absolute (25 kPa) to 350 mm Hg absolute (46.7 kPa), typically 30 to 31 kPa. Once the desired esterification reaction temperature is reached, the energy input to the reaction mixture is reduced to a level below the optimum level for reactivity to reduce the foaming tendency of the mixture.

The total amount of catalyst that should be used is determined primarily by four factors. First, the total reaction rate generally increases as the amount of catalyst, typically, expressed in weight percent of catalyst per weight of limiting reactant, increases up to a certain optimal concentration. The reaction rate also depends on the particular catalyst activity, the reaction temperature, and the water content of the reaction mixture. A relatively high concentration of catalyst may result in the organometallic complex esterification catalyst reacting with itself, to form unreactive agglomerated catalyst. Furthermore, a relatively higher concentration of certain esterification catalysts can cause product haze formation. In addition, process economics dictate that beyond an optimal point, further catalyst addition is not economical. If the reaction mixture contains an appreciable amount of certain cationic species, then the catalyst requirement must be increased to reach a desired reaction rate. The amount of catalyst used will therefore be chosen having taken all these factors into consideration.

When no reflux drying is performed but even when a reflux drier and/or flash step is provided, stopping the reflux to the reactor before the end of the run is reached, drives the reaction faster to completion because no more water is returned to the reactor and at the same time the amount of excess reagent in the crude ester is reduced, such as down to 12-15 wt %, thereby reducing the volume of crude ester to be further processed and the amount of excess reagent that needs to be removed in the downstream finishing steps. In embodiment, we therefore prefer to stop the reflux at least 2 minutes, preferably at least 5 minutes, more preferably at least 7 minutes and even more preferably at least 10 minutes and even 15 minutes before the expected batch termination time. We have found that when the alcohol reflux is continued till the end of the batch and through a reflux drier, the water content of the crude ester at the end of the reactor run may still be as high as 50 ppm wt. When stopping the alcohol reflux about 12 minutes before the end of the batch termination time, the water content of the crude ester may be only 20 ppm wt., or may even reach 10 ppm wt. or below. We have found that the presence of water, even in these small amounts, may have a surprisingly large effect on the rate of the reaction at the end of run, and therefore on the completion of the reaction and on the total batch time.

When the reflux to the reactor, or to the reflux drying step, is stopped, the alcohol coming from the overhead collection drum is routed to the recycle alcohol tank. Employing the techniques several embodiments of the present invention have been found to reduce the amount of acid or ester carried over in the reflux alcohol thus resulting in a purer product and requiring less separation techniques. When the batch is to be terminated, heat input to the reactor is stopped and the vacuum is broken, preferably by allowing nitrogen into the reactor system, more preferably into the reactor overhead system. This breaking of the vacuum is considered the moment of termination of the batch. As soon as the vacuum is broken, the reactor content may immediately be evacuated to a collection vessel in and from which it may be further processed. In case the next batch of product is of the same quality, the reactor is then ready for starting the new batch.

The esterification process of the present invention may also include one or more of the following steps: removal of excess reagent by nitrogen or steam stripping; addition of adsorbents such as alumina, silica gel, activated carbon, clay and/or filter aid to the reaction mixture following esterification before further treatment. In certain cases, adsorbent treatment may occur later in the process, following stripping, and in still other cases the adsorbent step may be eliminated from the process altogether. Addition of water and base to simultaneously neutralize the residual organic acids and hydrolyze the catalyst (if present); filtration of solids from the ester mixture containing the bulk of the excess reagent (acid or alcohol) used in the esterification process; removal of the excess reagent from the ester mixture by, for example, steam or nitrogen stripping under vacuum and recycling of the excess reagent to the reaction vessel; and, removing solids from the stripped ester in a final filtration, may also be included in the process.

Esterification catalysts that may be used include acid catalysts and organometallic catalysts. Organometallic esterification catalysts are preferred and include titanium, zirconium and tin catalysts such as titanium, zirconium and tin alkoxides, carboxylates and chelates. See U.S. Pat. No. 3,056,818. Titanium alkoxides are particularly useful.

Typical titanium alkoxides which can be used as catalysts include tetramethyl titanates, tetraethyl titanates, tetrapropyl titanates, tetra-isopropyl titanates, tetrabutyl titanates, tetrapentyl titanates, tetrahexyl titanates, tetraheptyl titanates, tetra-octyl titanates, tetranonyl titanates, tetradecyl titanates including tetra-2-propylheptyl titanate, tetradodecyl titanates, tetrahexadecyl titanates, tetra-octadecyl titanates, tetraphenyl titanates, and mixtures thereof. The alkoxy groups on the titanium atom can all be the same or they can be different, and their alkyl chains may be normal and/or branched, or mixtures thereof. The tin or zirconium counterparts of the above alcoholates can be substituted in whole or in part as catalysts. Tetra-isopropyl titanate (TIPT) is very suitable. Tetra-isooctyl titanates (TIOT) are also useful.

In a class of embodiments, the preferred acid or anhydride component comprises phthalic acid or anhydride, trimellitic acid or anhydride, aromatic carboxylic acids such as benzoic acid, cyclohexane mono- or poly carboxylic acids and adipic acid or anhydride.

### Isosorbide Plasticizers

In another class of embodiments of the invention, acids derived from photosynthetic microorganisms may be used to produce isosorbide plasticizers. The production of isosorbide plasticizers has been discussed in WO 2008/095571 and EP 2 114 952 A. The Roquette Company has also suggested developing isosorbide plasticizers from vegetable oils in ICIS Chemical Business, 5-11, March 2007, page 21. Linear acids and mixtures of linear acids, such as, acids having 1-20 carbon atoms and mixtures thereof, alternatively, 1-10 carbon atoms and mixtures thereof, for example, n-octanoic acid, n-decanoic acid, and mixtures thereof, may be used to produce isosorbide plasticizers. In an embodiment, the plasticizers are mixed n-octanoic acid and n-decanoic acids esters of dianhydrohexitols, for example, isosorbide. The dianhydrohexitols may be derived from glucose sugars or other suitable sugars. These plasticizers are advantageous because they offer at least one of better PVC compatibility, processing improvements, and lower pour points. Optionally, they may be used with a low amount of branched aliphatic acids, such as, for example, isononanoic acid, 2-ethylhexanoic acid, and mixtures thereof. An example is provide below:

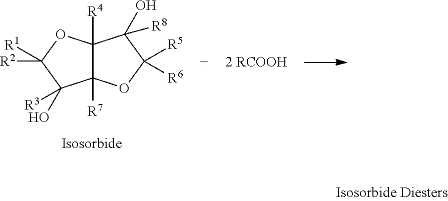

where R is a hydrocarbon radical having 1-24 carbon atoms, alternatively, 1-12 carbon atoms, alternatively, 1-10 carbon atoms.

### Salicylic Acid Esters

In another class of embodiments of the invention, salicylic acid esters may be produced from acids and alcohols derived therefrom from photosynthetic microorganisms. For example, acids and mixtures of acids, such as, acids having 1-20 carbon atoms and mixtures thereof, alternatively, 1-10 carbon atoms and mixtures thereof, optionally, linear, branched, or mixtures thereof. Examples include octanoic acid, decanoic acid, and mixtures thereof may be derived from photosynthetic microorganisms. In any of the embodiments described herein, the acids may be converted to their corresponding alcohols. Salicylic acid may be obtained from the bark of the willow tree or through known synthetic routes, for example, reacting phenol with caustic and carbon dioxide. These plasticizers are advantageous because they offer at least one of better PVC compatibility, processing improvements, and lower pour points. Optionally, they may be used with a low amount of branched aliphatic acids, such as, for example, isononanoic acid, 2-ethylhexanoic acid, and mixtures thereof. Additionally, performance improvements may be obtained by producing the salicylic acid esters by substituting at least some of the acids with an alcohol such as, for example, a phenol, benzyl alcohol, and mixtures thereof. An example is provided below:

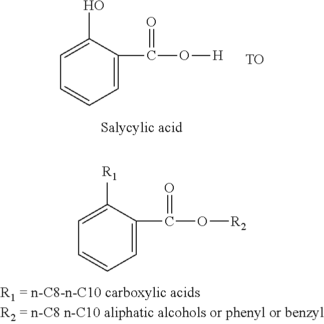

In an embodiment, salicylic acid esters may be prepared by reacting salicylic acid or 2-hydroxy benzoic acid with a mixture of n-octanol and n-decanol to prepare n-octyl n-decyl salicylate ester. The n-octyl n-decyl salicylate ester may then be reacted with n-octanoic acid and n-decanoic acid to make a diester. Optionally, to increase the reaction time, the acids may first be converted to acid halides, such as, an acid chloride, and then reacted with the salicylate ester.

In another alternative embodiment, the salicylic acid may be reacted with n-octanoic acid and n-decanoic acid to produce 2-n-octanoyl/n-decanoyl benzoic acid. The acid product may then be esterified with n-octanol and n-decanol by known esterification processes.

In yet another embodiment, phenyl or benzyl salicylate may be prepared from the esterification of salicylic acid with phenol or benzyl alcohol (or by the amine coupling reaction of salicylic acid and benzyl chloride) followed by the esterification of the hydroxyl group at the second position of the aromatic ring with a mixture of n-octanoic acid and n-decanoic acid derived from photosynthetic microorganisms.

### Hydrogenation

The plasticizer may also include a hydrogenated derivative of the esters described above. The hydrogenation of an ester to a hydrogenated derivative, such as phthalates to cyclohexanedicarboxylic acid esters or cyclohexanoates, is known. The terms may be used interchangeably unless otherwise distinguished. Generally, a process for hydrogenating an organic compound comprises bringing the organic compound into contact under hydrogenation conditions with a source of hydrogen in the presence of a catalyst comprising one or more catalytically active metal sites (optionally located on a catalyst support) and recovering the hydrogenated product.

For example, in an embodiment, di-n-octylphthalate (DnOP) may be hydrogenated into a hydrogenated derivative (DnOCH) wherein DnOP may be prepared by the esterification of hexahydrophthalic anhydride with 2 moles of n-octanol derived from a photosynthetic microorganism. It may also be prepared by the transesterification of dimethyl or diethyl hexahydrophthalate (also known as cyclohexane dicarboxylic acid ester of methanol or ethanol) wherein the n-octanol is derived from a photosynthetic microorganism.

General hydrogenation methods and materials may be found, for example, in DE 200 21 356, WO 99/32427, WO 03/029339, WO 2004/046076, WO 2004/046078, U.S. Pat. Nos. 7,297,738, 7,855,340, 7,413,813, 7,585,571, 7,595,420, 7,893,295, 7,732,634, and 7,875,742.

In a class of embodiments, the invention provides for a process for the production of a cyclohexanepolycarboxylic acid ester, comprising:

(a) obtaining at least one C₄ to C₁₃ alcohol derived from one or more photosynthetic microorganisms;

(b) contacting and reacting at least one carboxylic acid or at least one carboxylic acid anhydride with the at least one C₄ to C₁₃ alcohol to produce a product comprising an ester; and

(c) hydrogenating the ester to produce a second product comprising the cyclohexanepolycarboxylic acid ester.

In several embodiments, the hydrogenating comprises contacting the ester with a hydrogen-containing gas in the presence of a catalyst, the catalyst comprising one or more catalytically active metals applied to a catalyst support comprising a mixed porosity material containing mesopores and macropores.

The metal referenced above may be selected from the group consisting of at least one of platinum, rhodium, palladium, cobalt, nickel, ruthenium, and mixtures thereof

### Plasticizers and Blends of Plasticizers

The esters and their hydrogenated derivatives produced by the processes of the present invention, particularly, the phthalates and cyclohexanedicarboxylic acid esters, are particularly useful as plasticizers for polyvinyl chloride (PVC), polyurethane, and acrylic compositions.

In a class of embodiments, the esters and their hydrogenated derivatives produced by the processes of the present invention may be used, for example, as blends with other plasticizers. For a listing of other plasticizers that are suitable in blends, see, for example, U.S. Pat. No. 7,297,738. Additionally, the esters and their hydrogenated derivatives produced by the processes of the present invention may also be used with commercial phthalates, alkyl phthalates, and dialkyl phthalates, commercial cyclohexane dicarboxylic acids esters, citrates, terephthalates, adipates, trimellitates, benzoates, di-benzoates, and mixtures thereof.

For example, C₉ to C₁₁ alkyl benzoates, in particular the C₁₀ alkyl benzoate disclosed in U.S. Pat. No. 7,629,413, may be used with the esters and/or their hydrogenated derivatives.

Plasticizers in accordance with several embodiments of the invention will have 55.0 wt % or more biobased content, alternatively, 60.0 wt % or more biobased content, and alternatively, 65.0 wt % or more biobased content, based upon the total weight the plasticizer (ASTM D6866-10). In another embodiment, the plasticizer is about 66.6 wt % biobased content, based upon the total weight of the plasticizer (ASTM D6866-10). As used herein, “biobased content” refers to the amount of biobased carbon in the product as a percent of the weight (mass) of the total organic carbon in the product.

In accordance with several embodiments of the invention, the hydrogenated derivative or cyclohexanedicarboxylic acid esters may exhibit improvements in formulations. For example, the DnOCH (referenced above) made from n-octanol derived from photosynthetic microorganisms will offer advantages in PVC formulations as compared to two commercial cyclohexane dicarboxylic acid esters. In particular, DnOCH in accordance with an embodiment of the invention would offer one or more of the following: efficiencies in lower formulation costs and be easier to process than commercial DINCH material, exhibit reduced volatility compared to the 2-ethylhexanol version, exhibit superior low temperature properties compared to both products, and have superior UV resistance compared to both products.

In accordance with some embodiments, polymeric formulations made from the plasticizers of the invention may have greater acceptance in the market place because of “green” building requirements and the benefits associated with products carrying “green” labels or labels indicating “renewable content” of starting raw materials.

In particular, plasticizers in accordance with several embodiments of the invention may qualify for environmental credits such as LEEDS.

The esters and their hydrogenated derivatives optionally blended with other plasticizers find utility in toys, medical applications, medical films, bottle caps, food applications, food films, wire jacketing, insulation, coatings, vinyl sheet flooring, tarpaulins, shower curtains, table cloths, office supplies, packaging films, shoes, raincoats, mattress covers, child care articles, and the like.

### Examples

The following test methods are used in the examples.

### Hot Bench Gelation Temperature

The Hot Bench Gelation Temperature (HBG) is the temperature at which a defined plastisol layer forms a gel. HBG is measured on a metal gel block having a temperature gradient similar to a Geigy graded temperature gel block. A given quantity of plastisol is poured on the cold end of the block and drawn as a film along the length of the block. A stop-watch is started as soon as the film is drawn. After 60 seconds a strip of Mylar foil is laid over the plastisol film. The Mylar foil is removed and the point where no plastisol is adhering to the foil is marked. The temperature at the mark is recorded as the “HBG temperature”.

### Brookfield Viscosity

ASTM D 1824—Standard test method is used for apparent viscosity of plastisols and organosols at low shear rates using a Brookfield viscometer, spindle RV 1 to 7.

### Plasticizer Neat Viscosity and Density

ASTM D 7042—Standard Test Method is used for Dynamic Viscosity and Density of Liquids by Stabinger Viscometer (and the Calculation of Kinematic Viscosity).

### Low Temperature Flexibility

Clash & Berg measurement is used and based upon ASTM D 1043-84—Stiffness properties of plastics as a function of temperature by means of a torsion test.

### Neat Plasticizer Volatility

Neat plasticizer weight loss (in wt %) is measured on neat plasticizer after heating 10 g of plasticizer for 24 h at 115° C. in a forced ventilated oven (>160 air renewal/hour).

Some embodiments of the present invention are illustrated by reference to the following examples. It is to be understood that while the invention has been described in conjunction with the specific embodiments thereof, the foregoing description is intended to illustrate and not limit the scope of the invention. Other aspects, advantages and modifications will be apparent to those skilled in the art to which the invention pertains.

Therefore, the following examples are put forth so as to provide those skilled in the art with a complete disclosure and description and are not intended to limit the scope of that which the inventors regard as their invention.

In the Tables below, the various components mentioned are as follows:
Solvin™ 382 NG=an emulsion PVC available from Solvin
Jayflex™ DNP=di-isononyl phthalate available from ExxonMobil Chemical Company
DINCH=Hexamoll™ DINCH, di-isononyl cyclohexanoate available from BASF
DOCH=1,2-Cyclohexanedicarboxylic acid-bis-(2-ethylhexyl)-ester formerly available from Celanese Chemical
Baerostab™ CT9183XRF=a CaZn Standard, low phenol, 2-EHA free, nonylphenol free,
standard stabiliser available from Baerlocher
n-C8 cyclohexanoate=di-n-octyl cyclohexanoate (inventive)
n-C10 cyclohexanoate=di-n-decyl cyclohexanoate (inventive)
n-C8/n-C10 cyclohexanoate=mixture of di-n-octyl cyclohexanoate and di-n-decyl cyclohexanoate derived from a 50%/50% mix of n-octyl and n-decyl alcohols (inventive)

The above plasticizers, labelled as n-C8, n-C10 and nC8/-nC10 cyclohexanoates were obtained by conventional lab esterification of commercial cyclohexanedicarboxylic anhydride obtained from Acros Organics, with respectively n-octyl and n-decyl alcohol obtained from Acros Organics. Although the alcohols used to make the cyclohexanoates were not derived from photosynthetic microorganisms, the alcohols are believed to be representative of embodiments of the present invention as they possess similar chemical properties to those alcohols derived from photosynthetic microorganisms, for example, being linear molecules. The synthesis of di-n-octyl cyclohexanoate was obtained by esterification of cyclohexanedicarboxylic anhydride and n-octanol using 1% of TIOT (Tetra Iso Octyl titanate) from Tyzor and with 25% excess alcohol. The synthesis of di-n-octyl cyclohexanoate was obtained by esterification of cyclohexanedicarboxylic anhydride and n-octanol using 1% of TIOT (Tetra Iso Octyl titanate) from Tyzor and with 25% excess alcohol. The synthesis of di-n-octyl/di-n-decyl cyclohexanoate was obtained by esterification of cyclohexanedicarboxylic anhydride with an alcohol mix of 50/50 mol n-octanol/n-decanol using 1% TIOT (Tetra Iso Octyl titanate) from Tyzor and with 25% excess alcohol. For all such synthesis, the TIOT was added at 180° C. and the esterification reaction proceeded with conversion up to 99.9%. The crude ester was neutralized and stripped.

### Neat Properties of Synthesized Plasticizers

The inventive examples offer advantages over commercial cyclohexanoates, like DINCH or DOCH (see Table 1 below), due to their lower neat viscosity. A lower plasticizer viscosity results in a lower initial plastisol viscosity offering better plastisol rheology and easier processing.

In particular, the n-C8 cyclohexanoate exhibits a lower solution T° compared to DINCH, closer to the general purpose di-isononyl phthalate (DINP), a benchmark standard. This will result in faster gelation and improved processing of both plastisols and flexible PVC compounds.

Neat plasticizer volatility is significantly lower for the n-C10 cyclohexanoate and about 50% lower for the mix n-C8/n-C10 cyclohexanoates compared to commercial cyclohexanoates like DINCH. Lower volatility plasticizers result in lower plasticizer weight loss after heat ageing offering increased durability of finished flexible PVC articles.

Further, the n-C10 cyclohexanoate and the mix n-C8/n-C10 cyclohexanoates have lower density which results in volume cost advantages (each ton of a lower density plasticizer offers a greater volume of finished product or a lower cost per unit of volume).

TABLE 1
(Internal Reference - TSR 10-133/TSR 11-61)

| | nC8 | nC10 | Mix nC8/nC10 | | | |
|---|---|---|---|---|---|---|
| | cyclohexanoate | cyclohexanoate | cyclohexanoate | DOCH | DINCH | DINP |
| Dynamic viscosity in mPa · s | 28 | 39 | 34 | 45 | 50 | 94 |
| (at 20° C.) ASTM D7042 | | | | | | |
| Density (at 20° C.) in g/cm3 | 0.9497 | 0.9354 | 0.9416 | 0.9545 | 0.9466 | 0.972 |
| ASTM D7042 | | | | | | |
| Neat plasticizer weight loss in | 9.9 | 2.9 | 5.3 | | 9.6 | 5.8 |
| wt % (24 h at 115° C.) | | | | | | |
| Solution T (° C.) | 134 | 158 | 146 | | 141 | 128 |

### Plastisol Properties

To fulfil plastisol spread coating requirements in terms of production speed, PVC compositions are required to be of low viscosity and exhibit low gel temperature. The results presented in Table 2 show that the plastisol formulations derived from the n-C8 cyclohexanoate and the mix n-C8/n-C10 cyclohexanoates exhibit lower plastisol viscosity as shown from the Brookfield viscosity measurement.

Commercial cyclohexanoates like DINCH are known to exhibit very poor gelation and be difficult to process requiring higher processing temperatures. The hot bench gelation results listed in Table 2 clearly show that plastisols made from the n-C8 cyclohexanoates are faster gelling than DINCH and come closer to the gelation temperature of the general purpose di-isononyl phthalate based plastisol. Faster gelling plastisols are of particular interest in flooring, wall covering, UBC (e.g., underbody coating), and rotomolding (e.g., toys or playball production).

TABLE 2
(Plastisols Viscosity and Gelation)
Internal Reference: TSR 112/10 - Gelation Behavior (FU of TSR
3/10 and 34/10)

| Formulations | | | | | |
|---|---|---|---|---|---|
| Solvin 382 NG | | 100 | 100 | 100 | 100 |
| Jayflex DINP | | 60 | | | |
| DINCH | | | 60 | | |
| n-C8 cyclohexanoate | | | | 60 | |
| mix n-C8/n-C10 cyclohexanoate | | | | | 60 |
| Barostab CT9183XRF | | 1 | 1 | 1 | 1 |
| Brookfield viscosity | | | | | |
| mPa · s - spindle 3& 4 | | | | | |
| 2 h | mPa · s | 2600 | 1450 | 800 | 950 |
| 1 day | | 2800 | 1425 | 1000 | 925 |
| Hot bench gelation | ° C. | 96 | 130 | 99 | 135 |
| | | 98 | 129 | 104 | 135 |

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the invention, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All priority documents are herein fully incorporated by reference for all jurisdictions in which such incorporation is permitted and to the extent such disclosure is consistent with the description of the present invention. Further, all documents and references cited herein, including testing procedures, publications, patents, journal articles, etc. are herein fully incorporated by reference for all jurisdictions in which such incorporation is permitted and to the extent such disclosure is consistent with the description of the present invention.

While the invention has been described with respect to a number of embodiments and examples, those skilled in the art, having benefit of this disclosure, will appreciate that other embodiments can be devised which do not depart from the scope and spirit of the invention as disclosed herein.

1. A process for the production of an ester, comprising:
(a) obtaining at least one C₄ to C₁₃ alcohol derived from one or more photosynthetic microorganisms; and
(b) contacting and reacting at least one carboxylic acid or at least one carboxylic acid anhydride with the at least one C₄ to C₁₃ alcohol to produce a product comprising the ester resulting from the reaction.

2. The process of claim 1, wherein the at least one alcohol is at least one C₆ to C₁₁ alcohol.

3. The process of claim 1, wherein the at least one alcohol is at least one C₈ to C₁₀ alcohol.

4. The process of claim 1, wherein the at least one C₄ to C₁₃ alcohol comprises at least one of n-hexanol, n-octanol, n-decanol, and mixtures thereof.

5. The process of claim 1, wherein the carboxylic acid and the carboxylic acid anhydride are selected from the group consisting of at least one of phthalic anhydride, cyclohexanoic dicarboxylic acid or its anhydride, adipic acid or its anhydride, trimellitic acid or its anhydride, and mixtures thereof.

6. The process of claim 1, wherein the photosynthetic microorganisms are eukaryotic organisms.

7. The process of claim 1, wherein the photosynthetic microorganisms are selected from microalgae species.

8. The process of claim 1, wherein the photosynthetic microorganisms are selected from the group consisting of at least one of green algae (Chlorophyceae), yellow-green algae (Xanthophyceae), golden algae (Chrysophyceae), brown algae (Phaeophyceae), red algae (Rhodophyceae), diatoms (Bacillariophyceae), “pico-plankton” (Prasinophyceae and Eustigmatophyceae), and mixtures thereof.

9. The process of claim 1, wherein the at least one alcohol has a C¹⁴/C¹² ratio of 1:10¹².

10. The process of claim 1, wherein the at least one alcohol has a C¹⁴ content greater than zero.

11. A process for the production of a cyclohexanepolycarboxylic acid ester, comprising:
(a) obtaining at least one C₄ to C₁₃ alcohol derived from one or more photosynthetic microorganisms;
(b) contacting and reacting at least one carboxylic acid or at least one carboxylic acid anhydride with the at least one C₄ to C₁₃ alcohol to produce a product comprising an ester; and
(c) hydrogenating the ester to produce a second product comprising the cyclohexanepolycarboxylic acid ester.

12. The process of claim 11, wherein the hydrogenating comprises contacting the ester with a hydrogen-containing gas in the presence of a catalyst, the catalyst comprising one or more catalytically active metals applied to a catalyst support comprising a mixed porosity material containing mesopores and macropores.

13. The process of claim 12, wherein the metal is selected from the group consisting of at least one of platinum, rhodium, palladium, cobalt, nickel, ruthenium, and mixtures thereof.

14. The process of claim 11, wherein at least one of the catalytically active metal sites has been obtained via the partial or complete decomposition on the support of an organic complex of the metal.

15. A plasticizer composition comprising an ester of at least one C₄ to C₁₃ alcohol derived from one or more photosynthetic microorganisms and at least one carboxylic acid or at least one anhydride; wherein the plasticizer composition has a biobased content of 60.0 wt % or more (ASTM D6866-10) based upon the total weight of the plasticizer composition.

16. The plasticizer composition of claim 15, wherein the plasticizer composition has a biobased content of 65.0 wt % or more (ASTM D6866-10) based upon the total weight of the plasticizer composition.

17. The plasticizer composition of claim 15, wherein the plasticizer composition has a biobased content of about 66.6 wt % (ASTM D6866-10) based upon the total weight of the plasticizer composition.

18. The plasticizer composition of claim 15, wherein the ester is selected from C₆ to C₁₁ phthalates and mixtures thereof.

19. The plasticizer composition of claim 15, wherein the ester is selected from C₈ to C₁₀ dialkylphthalates and mixtures thereof.

20. The plasticizer composition of claim 15, wherein the ester is further hydrogenated to produce a cyclohexanepolycarboxylic acid ester.

21. The plasticizer composition of claim 15, wherein the plasticizer composition further comprises another plasticizer selected from the group consisting of at least one of phthalates (an additional phthalate if already present), cyclohexane dicarboxylic acids esters (an additional cyclohexane dicarboxylic acids ester if already present), citrates, terephthalates, adipates, trimellitates, benzoates, di-benzoates, and mixtures thereof.

22. A plasticized composition comprising the plasticizer composition of claim 15 and at least one polymer.

23. The plasticized composition of claim 22, wherein the at least one polymer is selected from at least one of polyvinyl chloride (PVC), polyurethane, acrylic compositions, and mixtures thereof.

24. The plasticized composition of claim 22, wherein the at least one polymer is polyvinyl chloride (PVC).

25. An article fashioned from the plasticized composition of claim 22 and selected from the group consisting of at least one of toys, medical applications, medical films, bottle caps, food applications, food films, wire jacketing, insulation, coatings, vinyl sheet flooring, tarpaulins, shower curtains, table cloths, office supplies, packaging films, shoes, raincoats, mattress covers, and child care articles.
